(19) United States
(12) Patent Application Publication
(10) Pub. No.: US 2025/0166176 A1
(43) Pub. Date: May 22, 2025

(54) METHODS AND MATERIALS FOR IMAGING ADIPOSE TISSUE

(71) Applicant: CellSight Technologies Inc., San Francisco, CA (US)

(72) Inventor: Jelena Levi, San Francisco, CA (US)

(73) Assignee: CellSight Technologies Inc., San Francisco, CA (US)

(21) Appl. No.: 18/841,542

(22) PCT Filed: Mar. 10, 2023
(86) PCT No.: PCT/US2023/064154

§ 371 Date: Aug. 26, 2024

Related U.S. Application Data
(60) Provisional application No. 63/318,923, filed on Mar. 11, 2022.

(51) Int. Cl. G06T 7/00; G16H 50/30

(52) U.S. Cl. CPC G06T 7/0012; G16H 50/30; G06T 2207/10104; G06T 2207/30024

(57) ABSTRACT

Embodiments of the present disclosure provide compositions and methods for performing positron emission tomography (PET) and, more particularly, to compositions and methods for the development and use of ¹⁸F-based PET tracers for use in selected imaging techniques such as studies of agents selected to modulate adipose tissue physiology.

### CROSS-REFERENCE TO RELATED APPLICATIONS

This application claims the benefit under 35 U.S.C. Section 119(e) of co-pending and commonly-assigned U.S. Provisional Patent Application Ser. No. 63/318,923, filed on Mar. 11, 2022, and entitled “METHODS AND MATERIALS FOR IMAGING ADIPOSE TISSUE” which application is incorporated by reference herein.

### BACKGROUND

Brown adipose tissue (BAT), with a high content of mitochondria, is associated with cardiometabolic health and has gained considerable attention because of a potential therapeutic effect in a range of conditions, from diabetes and obesity to cachexia in cancer (see, e.g., Becher, T., et al., Brown adipose tissue is associated with cardiometabolic health. Nat Med, 2021. 27(1): p. 58-65). In view of the potential therapeutic benefits associated with modulating the activation of BAT, processes associated with BAT activation and methods/drugs having the ability to modulate this activity are under investigation.

The instant disclosure relates, in general, to positron emission tomography (PET) and, more particularly, to compositions and methods for the development and use of ¹⁸F-based PET tracers for use in imaging adipose tissue such as brown adipose tissue. The long physical half-life (109 minutes) of ¹⁸F-based tracers allows for clinical studies without the necessity of an on-site cyclotron. In addition, with contemporary PET camera technology, quantitative measurements of adipose tissue radioactivity concentration can be made with high temporal sampling and good statistical precision.

The PET imaging agent most commonly used for imaging brown fat is 2-[F¹⁸]fluoro-2-deoxy-D-glucose (FDG). However, studies have shown that FDG does not image mitochondrial changes (most notably expression of UCP-1) but rather increase in glucose metabolism in activated brown fat (see, e.g. Hankir, M. K., et al., Dissociation Between Brown Adipose Tissue (18)F-FDG Uptake and Thermogenesis in Uncoupling Protein 1-Deficient Mice. J Nucl Med, 2017. 58(7): p. 1100-1103; and Hankir, M. K. and M. Klingenspor, Brown adipocyte glucose metabolism: a heated subject. EMBO Rep, 2018. 19(9)). Although activated brown fat indeed exhibits increased glucose metabolism, agents that affect glucose metabolism without activation of brown fat will concurrently lead to an increase in FDG in brown fat, rendering FDG a nonspecific agent for the activation of BAT.

Because FDG probes do not provide the specificity that is required to allow artisans to image the activation and/or deactivation of BAT in a manner that allows them to assess therapeutic agents and other therapeutic interventions, there is a need in the art for additional compositions and methods that are designed to use ¹⁸F-based PET tracers in techniques for adipose tissue imaging.

### SUMMARY

The compound used in the methods disclosed herein, [18F]-F-arabinofuranosyl guanine was initially developed as a PET imaging agent for activated T cells. This compound is a 18F-labeled analog of arabinofuranosyl guanine (AraG), one that can be phosphorylated, and trapped intracellularly, by two kinases: cytoplasmic deoxycytidine kinase (dCK) and deoxyguanosine kinase (dGK). As discussed below, we have discovered that, at tracer levels, [¹⁸F]F-AraG has the ability to be used in a number of new PET methodologies including those designed to observe the presence and/or activation of adipose tissue in vivo.

The disclosure provided herein is based in part upon on the discovery that [¹⁸F]F-AraG is a PET imaging agent that is well suited to observe the activation of adipose tissue cells. Embodiments of the invention include, for example, methods of imaging adipose tissue in a subject. Typically these methods include administering to the subject a compound having a formula:

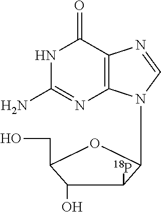

wherein a route of administration is selected so as to allow the compound to be phosphorylated by deoxycytosine kinase and deoxyguanosine kinase present in adipose tissue in the subject; and then imaging the subject via a positron emission tomography technique, wherein detecting the presence of the compound corresponds to the presence of adipose tissues and/or the activation of adipose tissues. In some embodiments of the invention, the adipose tissue is brown adipose tissue. In other embodiments of the invention, the adipose tissue is marrow adipose tissue (e.g., regulated marrow tissue).

In typical embodiments of the invention, these methods comprise observing the occurrence or levels of activated adipose tissue cells. In certain embodiments, the adipose tissue is present in a subject diagnosed with a pathological condition (e.g., obesity, diabetes, cachexia or a cancer such as a glioblastoma). In some embodiments of the invention, the subject is selected to be a patient that has been administered a therapeutic agent and/or undergone a therapeutic intervention (e.g., surgery, radiation, device implantation or the like), and the one or more images of the adipose tissue obtained are used to obtain information on the on the effects of the therapeutic agent or therapeutic intervention. While the methods disclosed herein can be used to observe the presence and/or activation of adipose tissue only once, optionally the method comprises observing one or more images of the adipose tissue on a first date; observing one or more images of the adipose tissue on a second date; and comparing the images obtained on the first date with the images obtained on the second date so as to observe changes in the adipose tissue over time.

Embodiments of the invention also include methods of observing the effects of a test agent on the activation of adipose tissue cells (e.g. brown adipose tissue cells, marrow adipose tissue cells and the like). Typically these methods comprise combining the adipose tissue cells with the test agent; observing the presence or absence of changes in the activation of the adipose tissue cells combined with the test agent; and then comparing the activation of the adipose tissue cells combined with the test agent with control adipose tissue cells that have not been combined with the test agent such that the effects of the test agent on the activation of adipose tissue cells is observed. In some embodiments of the invention, the adipose tissue cells are combined with the test agent in vitro, while in others, the adipose tissue cells are combined with the test agent in vivo. In certain embodiments of the invention, the adipose tissue cells are obtained from a subject diagnosed with a pathological condition (e.g., obesity, diabetes, cachexia or cancer).

Other objects features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 provides a schematic of the mechanisms involved in imaging adipose tissues with [¹⁸F]F-AraG.

FIG. 2 provides PET images showing that [¹⁸F]F-AraG shows a much higher accumulation in brown fat (yellow arrows) at day 23 (right image) post glioblastoma multiforme (gbm) cell implantation than at day 14 (left image).

FIG. 3 provides PET images showing activation of brown fat with [¹⁸F]F-AraG (upper panel images) and FDG (lower panel images). [¹⁸F]F-AraG shows BAT signal (yellow arrows) only in animals treated with BRL37344, a BAT activator, while FDG shows signal of different extent in all animals, indicating low specificity. Moreover, FDG showed an increase in signal in BAT in mice treated with insulin that affects only glucose metabolism. The data presented in this figure clearly show [¹⁸F]F-AraG's specificity and advantages over the FDG for imaging activated BAT.

FIG. 4 provides PET images showing that mice exposed to chronic dosing with BRL37344, a BAT activator, show an increase in [¹⁸F]F-AraG accumulation not only in intrascapular BAT but also in lumbar spine and proximal tibia (yellow arrows).

FIG. 5 provides PET images and data showing BAT activation is associated with T cell neuroinflammation in multiple sclerosis (cuprizone—EAE model). Treatment with fingolimod reduces neuroinflammation and leads to no activation of BAT.

FIG. 6 provides PET images showing simultaneous imaging of activated lymphocytes and adipocytes with [18F]F-AraG reveals concomitant neuroinflammation (A) and BAT activation (B) in a COVID-recovered patient.

### DETAILED DESCRIPTION

Before the present disclosure is described in greater detail, it is to be understood that this disclosure is not limited to particular embodiments described, and the embodiment of the invention as such may, of course, vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs. All publications and patents cited in this specification are herein incorporated by reference as if each individual publication or patent were specifically and individually indicated to be incorporated by reference and are incorporated herein by reference to disclose and describe the methods and/or materials in connection with which the publications are cited. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible. Unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text (e.g. U.S. patent Publication Nos. 20150230762, 20150297760 and 20190054198), in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

In accordance with the present disclosure, “a detectably effective amount” of embodiments of the present disclosure is defined as an amount sufficient to yield an acceptable image using equipment that is available for clinical use. A detectably effective amount of the embodiments of the present disclosure may be given in one or more administrations. The detectably effective amount of embodiments of the present disclosure can vary according to factors such as the degree of susceptibility of the individual, the age, sex, and weight of the individual, idiosyncratic responses of the individual, the dosimetry, and the like. Detectably effective amounts of embodiments of the present disclosure can also vary according to instrument and film-related factors. Optimization of such factors may bel within the level of skill in the art.

The term “detectable” refers to the ability to detect a signal or presence of an embodiment of the present disclosure over a background signal. The term “detectable signal” or the phrases “detection of a labeled compound” or “detectable labeled compound” refers to the detection (directly or indirectly) of a labeled compound in a host or sample. The detection of a labeled compound refers to the ability to detect and distinguish the presence of a labeled compound in a host or sample from other background signals derived from the host or sample. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background (e.g. in methods designed to assess adipose tissue activation). The detectable signal can be generated from a small to large concentration of a labeled compound. In an embodiment, the detectable signal may need to be the sum of each of the individual labeled compound signals. In an embodiment, the detectable signal can be generated from a summation, an integration, or other mathematical process, formula, or algorithm. In an embodiment, the summation, the integration, or other mathematical process, formula, or algorithm can be used to process the detectable signal so that the detectable signal can be distinguished from background noise and the like.

As used herein, “agent”, “active agent”, or the like, can include a compound (e.g., labeled compound) of the present disclosure. The agent can be disposed in a composition or a pharmaceutical composition. As used herein, “pharmaceutical composition” refers to the combination of an active agent with a pharmaceutically acceptable carrier. As used herein, a “pharmaceutical composition” refers to a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The term “unit dosage form,” as used herein, refers to physically discrete units suitable as unitary dosages for human and/or animal subjects, each unit containing a predetermined quantity of a compound calculated in an amount sufficient (e.g., weight of host, disease, severity of the disease, etc.) to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the host.

The term “effective amount” as used herein refers to that amount of an embodiment of the present disclosure (which may be referred to as a labeled compound) being administered that can be used to image a cell such as a adipose cell. By “administration” is meant introducing an embodiment of the present disclosure into a subject. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used.

As used herein, the term “host” or “subject” includes humans, mammals (e.g., cats, dogs, horses, etc.), and other living animals. In particular, the host is a human subject. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use as a “sample”, such as mammalian (particularly primate such as human) blood, urine, or tissue samples, or blood, urine, or tissue samples of the animals mentioned for veterinary applications.

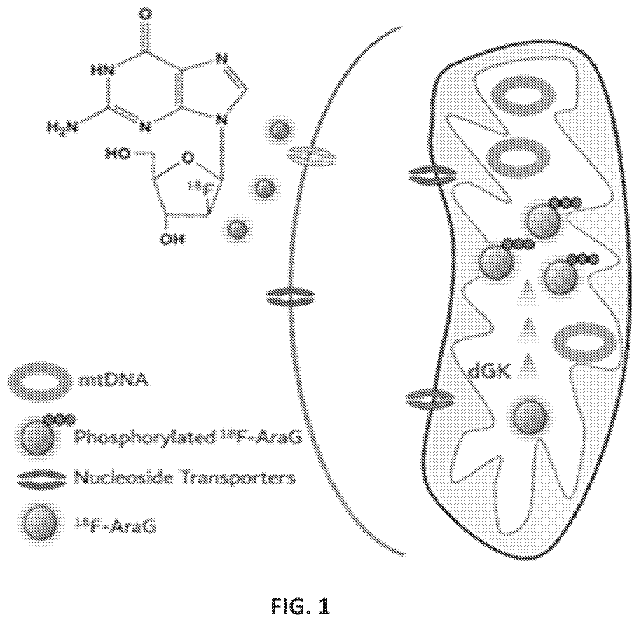

¹⁸F-AraG, also known as VisAcT, was developed as a PET tracer intended for imaging activated T cells in-vivo. [¹⁸F]F-AraG is an ¹⁸F-labeled analog of arabinofuranosylguanine (AraG), a compound that has shown selective accumulation in T cells. Nelarabine, AraG's prodrug, is US Food and Drug Administration (FDA) approved for treatment of T cell acute lymphoblastic leukemia and T cell lymphoblastic lymphoma. [¹⁸F]F-AraG enters T cells via nucleoside transporters and is trapped intracellularly through rate-limiting phosphorylation primarily by deoxyguanosine kinase (dGK), an enzyme, present solely in mitochondria, and critical in supplying nucleotides for mitochondrial DNA synthesis. Phosphorylation by dGK leads to entrapment and potential downstream accumulation into mtDNA allowing visualization via PET imaging (FIG. 1). [¹⁸F]F-AraG has already shown great promise in evaluating T cell involvement in graft versus host disease (GVHD), rheumatoid arthritis, cancer and multiple sclerosis (see, e.g. Ronald, J. A., et al., A PET Imaging Strategy to Visualize Activated T Cells in Acute Graft-versus-Host Disease Elicited by Allogenic Hematopoietic Cell Transplant. Cancer Res, 2017. 77(11): p. 2893-2902; Franc, B. L., et al., In Vivo PET Imaging of the Activated Immune Environment in a Small Animal Model of Inflammatory Arthritis. Mol Imaging, 2017. 16: p. 1536012117712638; Levi, J., et al., Imaging of Activated T Cells as an Early Predictor of Immune Response to Anti-PD-1 Therapy. Cancer Research, 2019. 79(13): p. 3455-3465; Levi, J., et al., (18)F-AraG PET for CD8 Profiling of Tumors and Assessment of Immunomodulation by Chemotherapy. J Nucl Med, 2021. 62(6): p. 802-807; and Guglielmetti, C., et al., Longitudinal imaging of T-cells and inflammatory demyelination in a preclinical model of multiple sclerosis using (18)F-FAraG PET and MRI. J Nucl Med, 2021).

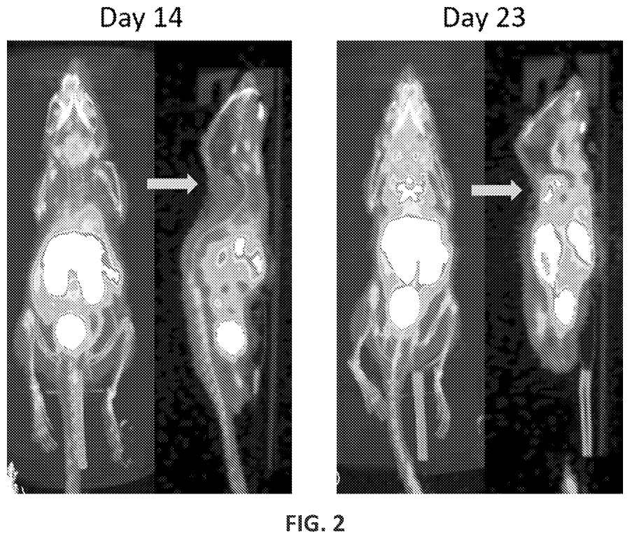

During our studies into the utility of ¹⁸F-AraG in assessing intracranial T cell infiltration in glioblastoma multiforme (GBM) we made the unexpected and surprising discovery that ¹⁸F-AraG can also image and/or assess the activation of adipose tissue (FIG. 2). Adipose, or fat, tissue (AT) was once considered an inert tissue that primarily existed to store lipids, and was not historically recognized as an important organ in the regulation and maintenance of health. With the rise of obesity and more rigorous research, AT is now recognized as a highly complex metabolic organ involved in a host of important physiological functions, including glucose homeostasis and a multitude of endocrine capabilities. AT dysfunction has been implicated in several disease states, most notably obesity, metabolic syndrome and type 2 diabetes. The study of AT can provide useful insights in developing strategies to combat these highly prevalent metabolic diseases.

Adipocytes are highly active endocrine cells that play a central role in overall energy homeostasis and are important contributors to some aspects of the immune system. They do so not only by influencing systemic lipid homeostasis but also through the production and release of a host of adipocyte-specific and adipocyte-enriched hormonal factors, cytokines, and extracellular matrix components (commonly referred to as “adipokines”). Little attention has been given to the role of adipose tissue in infectious disease. However, the strong proinflammatory potential of adipose tissue provides evidence for an important role in the systemic innate immune response. Furthermore, the adipocyte serves as an important target for the intracellular parasite Trypanosoma cruzi, the cause of Chagas' disease. In chronic Chagas' disease, adipocytes may represent an important long-term reservoir for parasites from which relapse of infection can occur. In other cases, such as with certain subtypes of adenoviruses, infection with viral particles leads to long-term hyperplasia and hyperproliferation of adipocytes, associating these adenoviral infections with a high propensity for subsequent obesity. In this context, the study of AT can provide useful insights in developing strategies to combat these diseases as well. In addition, obesity dramatically modifies the adipose tissue microenvironment in numerous ways, including induction of fibrosis and angiogenesis, increased stem cell abundance, and expansion of proinflammatory immune cells. As many of these changes also resemble shifts observed within the tumor microenvironment, proximity to adipose tissue may present a hospitable environment to developing tumors, providing a critical link between adiposity and tumorigenesis.

Embodiments of the invention include, for example, methods of imaging adipose tissue in a subject. Typically these methods include administering to the subject a compound having a formula:

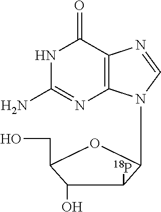

wherein a route of administration is selected so as to allow the compound to be phosphorylated by deoxycytosine kinase and deoxyguanosine kinase present in adipose tissue in the subject; and then imaging the subject, wherein detecting the presence of the compound corresponds to the presence of adipose tissue. In some embodiments of the invention, the adipose tissue is brown adipose tissue. In other embodiments of the invention, the adipose tissue is bone marrow adipose tissue (e.g., regulated bone marrow adipose tissue).

In certain embodiments of the invention, the methods are focused on imaging activated adipose tissues. In this context, a wide variety of aspects of the activation of adipose tissues are well know in the art and described, for example, in: Fenzi et al., Horm Mol Biol Clin Investig. 2014 July; 19(1):25-37; van der Vaart et al., Cells. 2021 May 6; 10(5):1122; Lim et al., Nat Protoc. 2012 Mar. 1; 7(3):606-15; Milton-Laskibar et al., J Physiol Biochem. 2020 May; 76(2):269-278; Bani Hassan et al., Curr Osteoporos Rep. 2019 December; 17(6):416-428; Tencerova et al., Best Pract Res Clin Endocrinol Metab. 2021 July; 35(4); and US Patent Application Nos. 20140088487, 20140199278, 20140324130, and 20180202000. In certain embodiments of the invention, the method comprises simultaneously imaging adipose tissues as well as T cells in the subject, and/or intracerebral infiltration of T cells in the subject; and/or the activation of brown adipose tissue and activation of bone marrow adipose tissue (e.g. regulated bone marrow adipose tissue) in the subject.

In certain embodiments of the invention, these methods comprise observing levels of activation in the adipose tissues that are associated with a pathology, for example, in situations where the subject is one diagnosed with a pathological condition (e.g., an infection such as a COVID-19 infection, a neuroinflammatory condition, a pathology exhibiting intracerebral lymphocyte activation or infiltration, obesity, diabetes, cachexia, or a cancer such as a glioblastoma). In some embodiments of the invention, the subject is selected to be a patient that has been administered a therapeutic agent and/or undergone a therapeutic intervention, and the one or more images of the adipose tissue obtained are used to obtain information on the on the effects of the therapeutic agent or therapeutic intervention.

In certain embodiments of the invention, the PET methods of imaging adipose cells in a subject responding to administration of a therapeutic agent further comprise observing one or more images obtained on a first date; observing one or more images on a second date; and then comparing the images obtained on the first imaging date with the images obtained on the second imaging date so as to observe changes in patient physiology over time that result from administration of the therapeutic agent (e.g. so as to distinguish between responders and nonresponders to the therapeutic agent). In certain embodiments of the invention, an amount of time from the first date to the second date comprises less than one week. Alternatively, an amount of time from the first date to the second date comprises at least one, two or three weeks, or at least one, two or three months.

In one illustrative working embodiment designed to show that [¹⁸F]F-AraG images activation of brown fat specifically, we treated mice with BRL37344 and insulin and imaged them using both [¹⁸F]F-AraG and FDG. BRL37344 is a b3 adrenergic receptor agonist that is known to activate BAT (see, e.g., Peng, X. R., et al., Unlock the Thermogenic Potential of Adipose Tissue: Pharmacological Modulation and Implications for Treatment of Diabetes and Obesity. Front Endocrinol (Lausanne), 2015. 6: p. 174) while insulin only affects glucose metabolism and does not affect activation of BAT.

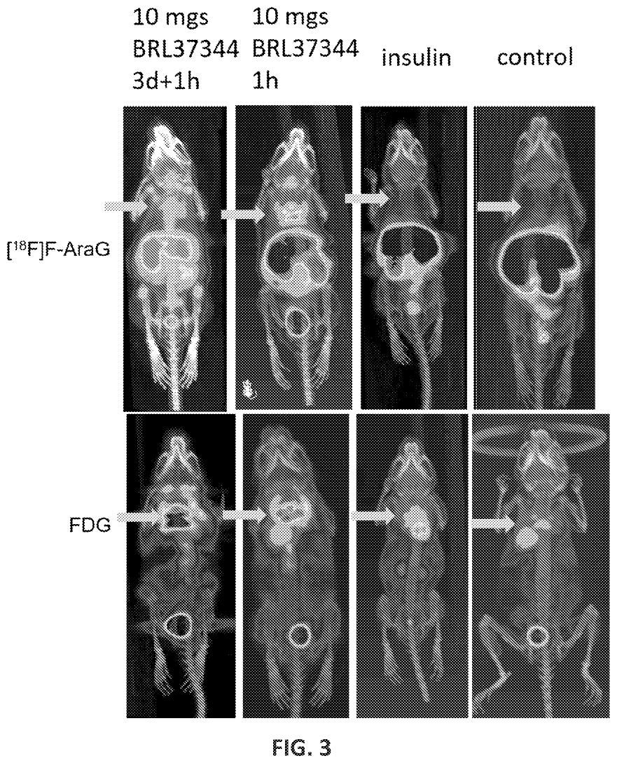

The results depicted in FIG. 3 clearly show [¹⁸F]F-AraG's specificity and advantage over the FDG for imaging activated BAT. While ¹⁸F]F-AraG showed activation of BAT only when BAT activator, BRL37344, was used, FDG showed an increase in signal in BAT of mice treated with insulin that affects only glucose metabolism. In addition, FDG shows signal in BAT in control animals as well, treated only with vehicle.

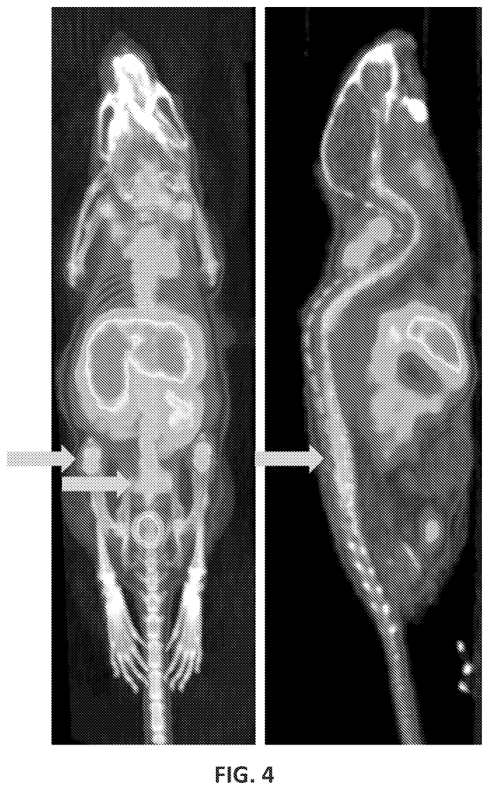

Furthermore, quite unexpectedly, yet very significantly, chronic dosing with BRL37344 (3 days+1 h prior to imaging) led to an increase in [¹⁸F]F-AraG signal not only in the classical, intrascapular BAT. but also in lumbar spinal cord and proximal tibia (FIG. 4).

This finding is of utmost importance as it is in these areas, lumbar spine and proximal tibia, that a special type of marrow adipose tissue (MAT) is found, that has similar properties to BAT such as response to cold conditions (see, e.g. Scheller, E. L., et al., Region-specific variation in the properties of skeletal adipocytes reveals regulated and constitutive marrow adipose tissues. Nat Commun, 2015. 6: p. 7808).

Overall, the ability of [¹⁸F]F-AraG to specifically image activation of BAT (and BAT-like MAT) can have a wide range of applications both in basic science and commercial. For example, embodiments of the invention can be used for drug development of agents that activate or deactivate BAT, and/or to follow a therapeutic regimen for diabetes, obesity or cachexia. Importantly, [¹⁸F]F-AraG can serve as an agent that can assess involvement of activated BAT in GBM and potentially bring new therapeutic approaches for GBM.

Embodiments of the present disclosure include methods of using [18F]-F-arabinofuranosyl guanine compounds, compounds that can be formed by a number of processes such as those disclosed in U.S. patent application Ser. No. 17/314,366 Filed on May 7, 2021 and entitled: METHODS AND MATERIALS FOR MAKING PET RADIOTRACERS, the contents of which are incorporated herein by reference. Such embodiments of the invention include, for example, [18F]-F-arabinofuranosyl guanine compounds formed from compositions of matter comprising a compound having the general formula:

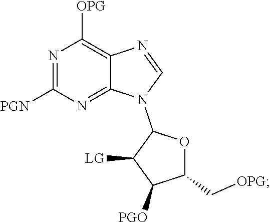

wherein: PG comprises a protecting group; and LG comprises a leaving group. Typically in such embodiments of the invention, the nitrogen atom coupled to the protecting group is coupled to two protecting groups as would be represented by “N(PG)₂”. Alternatively, this nitrogen atom is coupled to a hydrogen atom and a single protecting group as would be represented by “NHPG”. In illustrative embodiments of the invention, the composition includes at least one of precursor 1 or precursor 3:

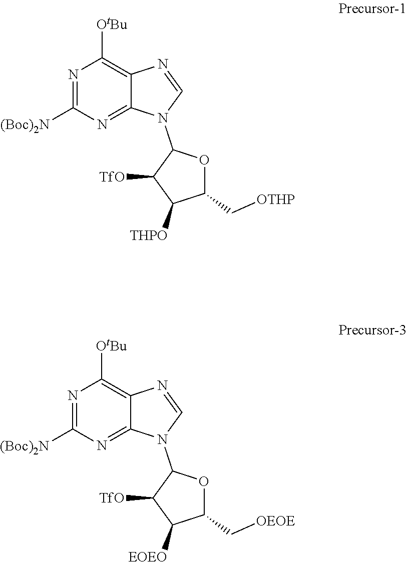

wherein: ^{t}Bu comprises a tert-Butyloxycarbonyl alcohol protecting group; Boc comprises a tert-Butyloxycarbonyl amine protecting group; THP comprises a tetrahydropyranyl alcohol protecting group; EOE comprises an ethoxyethyl alcohol protecting group; and Tf comprises a triflate leaving group. Embodiments of the present disclosure include methods of using [18F]-F-arabinofuranosyl guanine compounds formed by other processes such as those disclosed in US Application entitled “Compounds and Methods of Making Compounds”, having Ser. No. 17/185,502, filed on Feb. 25, 2021, the contents of which are incorporated herein by reference.

An illustrative embodiment of the method of imaging adipose cells, among others, includes: administering to the subject a compound of the present disclosure; and imaging the subject, wherein detecting the presence of the compound corresponds to the presence of the adipose cells. Another illustrative embodiment of the method of imaging the presence or extent of adipose metabolic activation, among others, includes: administering to the subject a compound of the present disclosure; and imaging the subject, wherein detecting the presence of the compound corresponds to the presence or extent of adipose cell activation in the subject.

Embodiments of this disclosure also include methods of imaging adipose tissues and cells. In general, embodiments of the labeled compounds can be used to image the localization and/or quantity of adipose cells in subjects (e.g., a living human). The labeled compounds can be administered to the subject and then the subject or a portion of the subject can be imaged using a device such as Positron Emission Tomography (PET) to detect the presence and location within the subject, and/or quantity of the labeled compounds present. The presence and/or quantity can be used to detect the presence, location, and/or number/size of adipose cells, cancer cells and/or leukocytes in the subject.

The present disclosure can also provides packaged compositions including the precursor compounds or intermediates to the labeled compounds instructions for making the labeled compounds and methods of use (e.g., written instructions for their use). The kit can further include appropriate buffers and reagents known in the art for administering embodiments of the present disclosure to a subject.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### EXAMPLES

### Example 1: [¹⁸F]F-ARAG Accumulation in Bat is Specific for Adrenergic Stimulation

In mice, regulation of the BAT by the SNS is predominately driven by 03 adrenergic receptor signaling (Cannon, B. and J. Nedergaard, Brown adipose tissue: function and physiological significance. Physiol Rev, 2004. 84(1): p. 277-359, Arch, J. R., et al., Atypical beta-adrenoceptor on brown adipocytes as target for anti-obesity drugs. Nature, 1984. 309(5964): p. 163-5). To examine [¹⁸F]F-AraG's accumulation in activated BAT, we treated mice with BRL37344, a well-studied b3 adrenergic receptor agonist (Cawthome, M. A., et al., BRL 35135, a potent and selective atypical beta-adrenoceptor agonist. Am J Clin Nutr, 1992. 55(1 Suppl): p. 252S-257S). Administration of BRL37344 one hour prior to imaging led to a considerable accumulation of [¹⁸F]F-AraG (10.98±1.64% ID/g) in intrascapular BAT (iBAT). Control animals as well as the animals that were treated with insulin showed significantly lower [¹⁸F]F-AraG uptake in iBAT (3.36±0.62% ID/g for insulin-treated and 4.11±0.76% ID/g for controls). In comparison, FDG, a tracer most commonly used for imaging BAT activation, showed an increased accumulation in iBAT not only with BRL37344 but in insulin-treated animals as well. These results provide evidence that, unlike FDG that shows adrenergically independent uptake in iBAT (Hankir, M. K. and M. Klingenspor, Brown adipocyte glucose metabolism: a heated subject. EMBO Rep, 2018. 19(9)), [¹⁸F]F-AraG accumulation indicates a process that is specific for adrenergic stimulation.

### Example 2: Chronic Adrenergic Stimulation Leads to Accumulation of [¹⁸F]F-Arag in the Bone Marrow (Adipocytes)

Surprisingly, chronic stimulation with BRL37344 resulted in [¹⁸F]F-AraG accumulation not only in iBAT, but also in the axilla, lumbar region and the bone marrow of the tibia and femur.

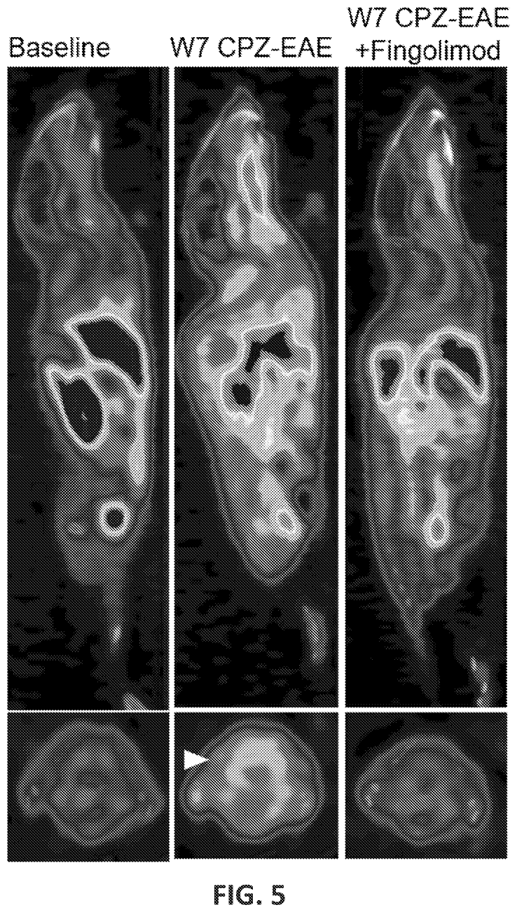

### Example 3: Activation of BAT is Associated with GBM Induced-Neuroinflammation

[¹⁸F]F-AraG ability to track stimulated adipocytes allowed observation of activated iBAT in mice with bioluminescently-tagged GBM tumors. The activation occurred at different time points and varied in intensity. In one mouse that had the highest neuroinflammation as evidenced by the intracerebral [¹³F]F-AraG signal the activation was the most dramatic, leading to a close to 9 fold increase in iBAT [¹⁸F]F-AraG signal (14.92% ID/g in OnTx3 vs. 1.70% ID/g in PreTx). In addition to the significant increase in signal in multiple brown fat depots, this mouse also showed a signal increase in the lumbar vertebrae as well as in the bone marrow of the proximal femur and tibia. See FIG. 5.

### Example 4: Activation of BAT is Associated with Neuroinflammation in Multiple Sclerosis

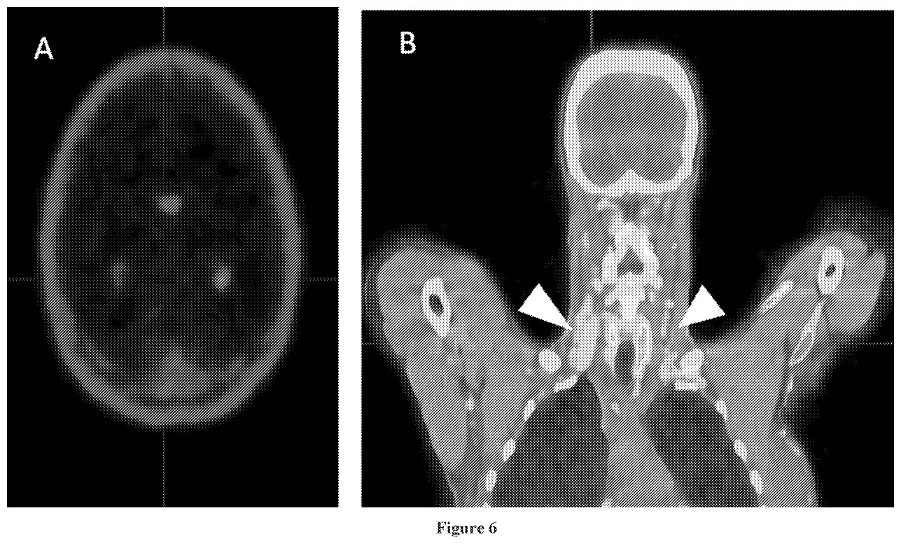

As the tumor in the brain represents an injury that can result in a stress response and BAT activation, we further investigated the relationship between BAT activation and inflammation in a mouse model of multiple sclerosis (MS). Evaluation of [¹⁸F]F-AraG signal in the iBAT in MS affected mice demonstrated that BAT activation coincides with intracerebral T cell infiltration. Furthermore, treatment with fingolimod, a drug that reduces intracerebral lymphocyte infiltration, led to a significant decrease in iBAT signal. See FIG. 6. Overall, these results provide evidence of an association of BAT activation with neuroinflammation.

### Example 5: [¹⁸F]F-ARAG can Detect Activated Brown Fat in Humans

Since neuroinflammation that includes T cells involvement has been linked to neurological problems experienced by some patients with acute and long COVID-19, we looked for the evidence of activated BAT in COVID-19-recovered patients. In contrast to virus unaffected healthy volunteer, both neuroinflammation and activated BAT were detected in a couple of patients who recovered from COVID-19. As shown in FIG. 7, the location of the [¹⁸F]F-AraG signal matched the sites of most commonly observed BAT depot in humans, cervical and supraclavicular (FIG. 7 F-H). These results show that, similar to what was observed in mice, [¹⁸F]F-AraG can simultaneously detect both neuroinflammation and BAT activation in humans.

### REFERENCES

1. Ronald, J. A., et al., A PET Imaging Strategy to Visualize Activated T Cells in Acute Graft-versus-Host Disease Elicited by Allogenic Hematopoietic Cell Transplant. Cancer Res, 2017. 77(11): p. 2893-2902.
2. Franc, B. L., et al., In Vivo PET Imaging of the Activated Immune Environment in a Small Animal Model of Inflammatory Arthritis. Mol Imaging, 2017. 16: p. 1536012117712638.
3. Levi, J., et al., Imaging of Activated T Cells as an Early Predictor of Immune Response to Anti-PD-1 Therapy. Cancer Research, 2019. 79(13): p. 3455-3465.
4. Levi, J., et al., (18)F-AraG PET for CD8 Profiling of Tumors and Assessment of Immunomodulation by Chemotherapy. J Nucl Med, 2021. 62(6): p. 802-807.
5. Guglielmetti, C., et al., Longitudinal imaging of T-cells and inflammatory demyelination in a preclinical model of multiple sclerosis using (18)F-FAraG PET and MRI. J Nucl Med, 2021.
6. Becher, T., et al., Brown adipose tissue is associated with cardiometabolic health. Nat Med, 2021. 27(1): p. 58-65.
7. Hankir, M. K., et al., Dissociation Between Brown Adipose Tissue (18)F-FDG Uptake and Thennogenesis in Uncoupling Protein 1-Deficient Mice. J Nucl Med, 2017. 58(7): p. 1100-1103.
8. Hankir, M. K. and M. Klingenspor, Brown adipocyte glucose metabolism: a heated subject. EMBO Rep, 2018. 19(9).
9. Peng, X. R., et al., Unlock the Thermogenic Potential of Adipose Tissue: Pharmacological Modulation and Implications for Treatment of Diabetes and Obesity.

Front Endocrinol (Lausanne), 2015. 6: p. 174.
10. Scheller, E. L., et al., Region-specific variation in the properties of skeletal adipocytes reveals regulated and constitutive marrow adipose tissues. Nat Commun, 2015. 6: p. 7808.

1. A method of imaging adipose tissue in a subject comprising:
(a) administering to the subject a compound having a formula: 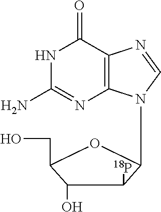
wherein a route of administration is selected so as to allow the compound to be phosphorylated by deoxycytosine kinase and deoxyguanosine kinase present in adipose tissue in the subject; and
(b) imaging the subject, wherein detecting the presence of the compound corresponds to the presence of adipose tissue.

2. The method of claim 1, wherein:
the adipose tissue is brown adipose tissue;
the adipose tissue is marrow adipose tissue; and/or
the adipose tissue is activated adipose tissue.

3. The method of claim 1 or claim 2, wherein the method further comprises observing levels of cellular activation in the adipose tissue, wherein levels of cellular activation in the adipose tissue are observed by observing amounts of the compound in the adipose tissue.

4. The method of claim 1, wherein the method further comprises simultaneously imaging:
T cells in the subject;
intracerebral infiltration of T cells in the subject; and/or
activation of brown adipose tissue and activation of regulated marrow adipose tissue in the subject.

5. The method of claim 1, wherein the subject is one diagnosed with a pathological condition.

6. The method of claim 5, wherein the pathological condition comprises an infection, a neuroinflammatory condition, a pathology exhibiting intracerebral lymphocyte activation, obesity, diabetes, cachexia or cancer.

7. The method of claim 6, wherein the pathological condition is COVID-19 infection, a glioblastoma, a pancreatic cancer or multiple sclerosis.

8. The method of claim 5, wherein the subject is selected to be a patient that has been administered a therapeutic agent and/or undergone a therapeutic intervention, and the one or more images of the adipose tissue obtained in step (b) are used to obtain information on the on the effects of the therapeutic agent or therapeutic intervention.

9. The method of claim 8, wherein the method further comprises observing one or more images of the adipose tissue on a first date; observing one or more images of the adipose tissue on a second date; and comparing the images obtained on the first date with the images obtained on the second date so as to observe changes in the adipose tissue over time.

10. The method of claim 9, wherein an amount of time from the first date to the second date comprises at least one day, at least one week or at least one month.

11. A method of observing the effects of a test agent on the activation of adipose tissue cells, the method comprising:
combining the adipose tissue cells with the test agent;
observing the presence or absence of changes in the activation of the adipose tissue cells combined with the test agent; and
comparing the activation of the adipose tissue cells combined with the test agent with control adipose tissue cells that have not been combined with the test agent such that the effects of the test agent on the activation of adipose tissue cells is observed.

12. The method of claim 12, wherein the adipose tissue cells are combined with the test agent in vitro.

13. The method of claim 12, wherein the adipose tissue cells are activated marrow adipose tissue cells.

14. The method of claim 12, wherein the adipose tissue cells are obtained from a subject diagnosed with a pathological condition.

15. The method of claim 14, wherein the pathological condition is a neuroinflammation, obesity, diabetes, cachexia or cancer.
